(19) United States
(12) Patent Application Publication
(10) Pub. No.: US 2007/0224088 A1
(43) Pub. Date: Sep. 27, 2007

(54) Fluid processing device including output interface with analyzer

(75) Inventors: David Maurice Cox, Foster City, CA (US); Abizar Lakdawalla, Cupertino, CA (US)

Correspondence Address: KILYK & BOWERSOX, P.L.L.C., 3603 CHAIN BRIDGE ROAD, SUITE E, FAIRFAX, VA, 22030, US

(73) Assignee: Applera Corporation, Foster City, CA (US)

(21) Appl. No.: 11/388,655

(22) Filed: Mar. 24, 2006

(51) Int. Cl. G01N 33/00

(52) U.S. Cl. 422/100; 436/174; 436/180; 422/63

(57) ABSTRACT

A fluid processing device, system, and method, are provided, for processing one or more samples. The device can comprise two or more sets of substantially parallel fluid processing pathways, each set comprising one or more fluid processing pathways. Each fluid processing pathway can comprise a reaction region, one or more inlet chambers, and one or more outlet chambers. The one or more outlet chambers of each set of fluid processing pathways can be arranged in an array configured to interface with an analyzer comprising a plurality of injectors, for example, injectors for capillaries of a multi-capillary electrophoresis instrument. The one or more inlet chambers of each set of fluid processing pathways can be arranged in an array configured to interface with a loading device comprising a plurality of injectors, for example, the loading tips of a multi-pipette robotic filling device.

### FIELD

The present teachings relate to fluid processing devices, systems, and methods for using such devices and systems. More particularly, the present teachings relate to devices that manipulate, process, or otherwise alter fluids and fluid samples.

### INTRODUCTION

Fluid processing devices are used for manipulating fluid samples. There continues to exist a demand for fluid processing devices, methods of using them, and systems incorporating them, for processing samples, that are fast, reliable, consumable, and can be used to process a large number of samples, for example, micro-sized samples, simultaneously.

### SUMMARY

According to various embodiments, a fluid processing device is provided that can comprise a substrate comprising a top surface and two or more sets of substantially parallel fluid processing pathways in communication with at least a portion of the top surface. Each set can comprise one or more fluid processing pathways. Each fluid processing pathway of a set can be provided adjacent to and vertically offset from, one or more fluid processing pathways of a different set. Each fluid processing pathway can comprise at least a first region, and one or more outlet chambers disposed downstream from the first region and in fluid communication with the first region. For a set that comprises multiple fluid processing pathways each comprising an inlet chamber, the inlet chambers can be configure to interface with an array of ends of a loading device, for example, a robotic filler. The one or more outlet chambers of a set of fluid processing pathways can be configured to interface with the ends of a processing device. The processing device can comprise a plurality of ends arranged in an array, for example, a plurality of injector ends of a multi-capillary electrophoresis analyzer.

According to various embodiments, a system is provided that can comprise a fluid processing device, as described herein, and a processing device that can comprise a plurality of ends arranged in an array. The inlet chambers of a set of fluid processing pathways of the device can be configured to interface with an array of ends of a loading device, for example, a robotic filler. The outlet chambers of a set of fluid processing pathways of the device can be configured to interface with the array of ends of the processing device.

According to various embodiments, a method is provided that can comprise providing a processing device that can comprise a plurality of ends arranged in a first array, and a fluid processing device that can comprise two or more sets of substantially parallel fluid processing pathways. Each set can comprise two or more fluid processing pathways. Each fluid processing pathway of a set can be disposed adjacent to and vertically offset from, a fluid processing pathway of a different set. Each fluid processing pathway can comprise at least a first region, and one or more outlet chambers disposed downstream from the first region and in fluid communication with the first region. The one or more outlet chambers of a set of fluid processing pathways can be arranged in a second array, and each set of the two or more sets of fluid processing pathways can define a different second array. The method can comprise moving one or both of the fluid processing device and the processing device relative to one another such that each end of the first array can be inserted into a complementary outlet chamber of an array defined by a set of fluid processing pathways. The same or a similar method can comprise moving one or both devices such that each tip of an array of loading tips can be inserted into a complementary inlet chamber of an array of inlet chambers.

According to various embodiments, a method is provided that can comprise flowing a liquid sample through one or more fluid processing pathways of a device as described herein, and into outlet chambers of the device, and then moving the device relative to one or more corresponding processing devices as described herein, such that each end of the first array of ends of the processing device can be inserted into a complementary outlet chamber of a second array that can be defined by a set of fluid processing pathways. Flowing the liquid sample can comprise, for example, centrifugally spinning the device.

According to various embodiments, the fluid processing system can comprise a fluid processing device and a multi-capillary electrophoretic analyzer. The fluid processing device can comprise a substrate comprising a top surface and two or more sets of substantially parallel fluid processing pathways. Each pathway can comprise at least one respective outlet chamber in communication with at least a portion of the top surface. Each set can comprise two or more fluid processing pathways. At least one of the outlet chambers of one of the sets of fluid processing pathways can be disposed between two outlet chambers of a different set of fluid processing pathways. The analyzer can comprise a set of injectors, wherein the number of injectors in the set of injectors can be the same as, or less than, the number of outlet chambers in each set of fluid processing pathways. The injectors of the set of injectors can comprise distal tips arranged in a first array. The outlet chambers of each set of fluid processing pathways can be arranged in an array that matches or has the same spacing as the first array.

It is to be understood that both the foregoing general description and the following detailed description are exemplary and explanatory only and are intended to provide a further explanation of the present teachings.

### DRAWINGS

The skilled artisan will understand that the drawings described below, are for illustration purposes only. The drawings are not intended to limit the scope of the present teachings in any way.

FIG. 1 is a top-plan view of a fluid processing device comprising fluid processing pathways according to some embodiments.

FIG. 2 is an enlarged top-plan view of several fluid processing pathways.

FIG. 3 is a perspective view of a fluid processing device comprising outlet chambers and a processing device comprising a plurality of ends that can interface with the outlet chambers of the fluid processing device, according to various embodiments.

It is to be understood that both the foregoing general description and the following detailed description are exemplary and explanatory only and are intended to provide a further explanation of the various embodiments of the present teachings.

### DESCRIPTION

According to various embodiments, a fluid processing device, for example, a microfluidic device, can be provided. The fluid processing device can comprise a substrate that can comprise a top surface and two or more substantially parallel fluid processing pathways provided in communication with at least a portion of the top surface. Each set can comprise one or more fluid processing pathways. Each fluid processing pathway of a set can be provided adjacent to and vertically offset from, a fluid processing pathway of a different set. Each fluid processing pathway can comprise at least a first region, and one or more outlet chambers disposed downstream from the first region and in fluid communication with the first region. The one or more outlet chambers of a set of fluid processing pathways can be configured to interface with a processing device, for example, an analyzer.

According to various embodiments, the fluid processing device can comprise one inlet chamber or a plurality of inlet chambers. For example, a proximal end of each fluid processing pathway can be in fluid communication with a single inlet chamber. Each fluid processing pathway can comprise an inlet chamber at its proximal end. The fluid processing device can comprise a cover provided over at least a portion of the top surface. The cover can comprise one or more access areas, wherein each access area can correspond to a region, for example, an outlet chamber, to form, for example, an accessible outlet chamber. An access area can allow access to a corresponding region, for example, an outlet chamber or an inlet chamber. In some embodiments, the fluid processing device can comprise a high density fluid processing device.

According to various embodiments, the fluid processing device can comprise a plurality of outlet chambers arranged in an array. For example, the fluid processing device can comprise 4, 8, 16, 24, 32, 48, 96, 192, and 384, or more outlet chambers arranged in an array. According to various embodiments, the outlet chambers can be arranged in any number of possible arrays utilizing varied or uniform spacing, and regular or irregular repeating or non-repeating geometric arrangements. For example, an array can comprise a square matrix, a diamond matrix, or a rectangular matrix where the points of the geometric shape are represented by features of the array.

According to some embodiments, the outlet chambers can be arranged in one single array on the device, or in a plurality of arrays. According to some embodiments, the outlet chambers can comprise or contain a product from a reaction performed in and/or on the fluid processing device, or can contain a product of one or more analyses, filtrations, separations, or the like, performed in and/or on the fluid processing device. The fluid processing device can comprise a plurality of fluid processing pathways. Each pathway can comprise at least one channel and a respective one or more output well. Samples can be processed as they pass through the pathways of the fluid processing device, and can be ready for further analysis when outputted to the outlet chambers. According to some embodiments, in a 192-outlet chamber fluid processing device, the outlet chambers can comprise, for example, a diameter of about 2.0 mm and can comprise, for example, a depth of about 1.5 mm. In a 384-outlet chamber fluid processing device, the outlet chambers can comprise, for example, a diameter of about 1.5 mm and can comprise, for example, a depth of about 1.5 mm.

In some embodiments, a fluid processing pathway can comprise, for example, one or more of a region, an area, an access area, a channel, a branch, and a valve. A region can comprise any shape or form capable of retaining a volume of fluid. For example, a region can comprise a surface area, an area, a volume, a recess, a chamber, a depression, a well, a through hole, a space, or the like. A region can comprise any shape, for example, round, teardrop, square, irregular, ovoid, rectangular, cubic, or the like. A region or channel can comprise any cross-section configuration, for example, square, round, ovoid, irregular, trapezoid, circular, semi-circular, or the like. For example, a channel can comprise a cross-sectional area that has an aspect ratio, that is, a width/depth ratio, of greater than one, less than one, or one. A channel can comprise a semi-oval cross-sectional area formed in a substrate. The cross-sectional area can comprise an aspect ratio, that is, a width/depth ratio, of greater than one, less than one, or one. In some embodiments, a channel can comprise a thin and narrow channel formed in a substrate, wherein the cross-sectional area has an aspect ratio, that is, a width/depth ratio, of less than one. A channel can comprise a trapezoidal cross-sectional area and generally can comprise an aspect ratio of less than one. These and other cross-sectional designs can be used as channels, for example, flow-restricting channels, and can be preformed or formed during a valve-opening operation according to various embodiments.

According to some embodiments, an inlet chamber of a fluid processing device can be teardrop-shaped, having a wide end and a narrow end, wherein the narrow end can be in fluid communication with, for example, a channel of a fluid processing pathway. Each of the regions described herein can comprise, for example, an inlet chamber, an outlet chamber, a reaction region, a purification region, a separation region, a processing region, a storage region, an incubation region, or the like. A reaction region can be provided, for example, between an inlet chamber and an outlet chamber. Inlet and outlet access areas can also be provided through for example, one or more of a top surface of a fluid processing device, through a bottom surface of the device, through a side edge or end edge of the device, through the substrate, through the cover layer, and through a combination of these features. For example, the device can comprise an inlet access area through a cover layer and in communication with an inlet chamber of the device. The device can comprise an outlet access area provided through a cover layer and in communication with an outlet chamber.

According to some embodiments, the one or more fluid processing pathways of the two or more sets, can be provided, for example, in a top surface of a substrate, on a top surface of a substrate, in a substrate, in a bottom surface of a substrate, on a bottom surface of a substrate, in an edge of a substrate, on an edge of a substrate, or in or on any combination thereof. Two or more of the outlet chambers of the two or more substantially parallel fluid processing pathways can be aligned and can define an axis substantially perpendicular to an axis defined by the substantially parallel fluid processing pathways.

In some embodiments, the one or more outlet chambers can comprise a first outlet chamber and a second outlet chamber. The first and the second outlet chambers can each comprise dead end outlet chambers. A dead-end outlet chamber can be provided last in a fluid processing pathway. For example, a fluid processing pathway can comprise a flow splitter that splits the flow path into two branch channels. A first branch channel can comprise a first outlet chamber disposed at its distal end, and a second branch channel can comprise a second outlet chamber disposed at its distal end.

According to various embodiments, one or more flow splitters for splitting the fluid sample from one sample into two or more samples or aliquots along two or more branch channels of a fluid processing pathway, can be provided in one or more of the one or more fluid processing pathways, for example, for splitting a sample into 2, 3, 6, 12, 24, 48, 96, 192, or 384 samples or aliquots. According to some embodiments, a flow splitter can be disposed downstream of an inlet chamber, to split the pathway into two or more branch channels or flow paths. Each branch channel can end at a respective, dead-end, outlet chamber or can be open-ended.

Branch channels can be used to obtain equal volumes of fluids in as many portions or aliquots as desired. Branch channels can be in fluid communication with a region, for example a processing region forming individual pathways for further processing of each aliquot. The pathways can be used to perform a single reaction or process, for example, forward sequencing, or can perform multiple same or multiple distinct reactions or processes, for example, PCR, on an aliquot. Reagents needed to perform a certain reaction or process in a reaction region of a pathway, can be loaded in the respective reaction region at the time of manufacture of the fluid processing device, or can be loaded at the time of use.

According to various embodiments, a branch channel or region can comprise one or more reagents disposed therein such that a reaction can take place in a branch channel or region. Reagents can be disposed in a branch channel or region, for example, a reaction region, using any methods known in the art. For example, reagents can be sprayed and dried, delivered using a diluent, injected using a capillary, a pipette, and/or a robotic pipette, or otherwise disposed in a region or channel of a fluid processing pathway. Branch channels can be provided substantially parallel to each other, not substantially parallel to each other, or can be provided at an angle relative to each other. Branch channels can be linear, substantially linear, curved, a combination of any two or more thereof, or the like.

In some embodiments, at least one of the one or more fluid processing pathways can comprise a branched, substantially linear, fluid processing pathway. A fluid processing pathway can comprise a pathway including at least an inlet chamber and one or more outlet chambers. A fluid processing pathway can comprise one or more storage regions. A storage region can be provided upstream from and adjacent to, an outlet chamber of a pathway. A storage region of one or more fluid processing pathways can be in fluid communication with a corresponding outlet chamber. When an access area corresponding to an outlet chamber, comprises a slit in the cover, the fluid processing pathway can comprise a storage region provided upstream from and adjacent to, the outlet chamber.

In some embodiments, a fluid processing pathway can comprise a single storage region or a plurality of storage regions, wherein each storage regions can be in fluid communication with a corresponding outlet chamber of a fluid processing pathway or a branch channel of a fluid processing pathway. For example, a first storage region can be disposed upstream from and adjacent to a first outlet chamber of a first branch channel of a fluid processing pathway, and a second storage region can be disposed upstream from and adjacent to a second outlet chamber of a second branch channel of the fluid processing pathway. Pathways that can be used in the fluid processing device include those disclosed in U.S. Patent Application Publication No.: 2004/0018116 A1, to DESMOND, et al., filed Jan. 3, 2003, hereby incorporated by reference herein, in its entirety.

According to some embodiments, one or more of the one or more fluid processing pathways can further comprise one or more valves. The fluid processing device can comprise a series of regions that can be in fluid communication with adjacent regions or can be blocked from adjacent regions using, for example, a valve provided between adjacent regions of a fluid processing pathway. A valve can be disposed between adjacent regions to control fluid flow through the fluid processing pathway. A valve can comprise any material, structure, or configuration, which is capable of controlling fluid movement through a pathway, channel, region, or area, upon actuation. The valve can comprise a valve that can be opened, or can be opened and closed. The valve can comprise one or more valves that can be actuated by one or more of, for example, pressure, deformation, solubilization, pH change, cutting, heat, and force. In some embodiments, a valve can be provided between a storage region and a corresponding outlet chamber. According to various embodiments, the one or more valves can comprise one or more of an optical valve, a dissolvable valve, a heat-meltable valve, a pressure-actuated valve, a mechanical valve, a pH sensitive valve, and a deformable valve, for example, an intermediate wall. The deformable valve and devices for actuating such a valve can comprise those disclosed in United States Patent Application Publication No.: 2004/0131502 A1, to COX, et al., filed Mar. 31, 2003, hereby incorporated by reference in its entirety, herein. Other valves that can be used in the fluid processing device can comprise those disclosed in U.S. Pat. No.: 6,817,373 B2, to COX, et al., issued Nov. 16, 2004, and U.S. Patent Application Publication No.: 2004/0055956 A1, to HARROLD, Michael, P., filed Jul. 28, 2003, each of which is hereby incorporated herein in its entirety.

According to various embodiments, one or more outlet chambers of a set of two or more sets of fluid processing pathways can be configured to interface with ends of a processing device comprising a plurality of ends arranged in an array. A processing device can comprise, for example, a standard 1, 4, 16, or 96 capillary device, for example, a standard 1, 4, 16, or 96 capillary ABI sequencing array, as described herein. A processing device can comprise those available from Applied Biosystems, Foster City, Calif. A series of inlet chambers can be configured to interface with a device for loading a sample or reagent, for example, a standard multi-channel pipette. Each aligned outlet chamber of a set of fluid processing pathways can be spaced equidistant from another. For example, each aligned outlet chamber can be spaced at from about 0.5 mm to about 20 mm apart, from about one mm to about 15 mm apart, from about two mm to about 12 mm apart, or at about nine mm apart. A fluid processing pathway can branch and can comprise, for example, an outlet chamber provided at a distal end of each branch channel, for example, a closed-end channel branch.

According to various embodiments, a first outlet chamber of a first branch channel can be vertically offset from a second outlet chamber of a second branch channel, wherein a first outlet chamber of each of a plurality of substantially parallel fluid processing pathways, form a first set of aligned outlet chambers, and a second outlet chamber of each of the plurality, form a second set of aligned outlet chambers. Two or more branch channels of a fluid processing pathway, can be provided substantially parallel to each other, not substantially parallel to each other, or at an angle relative to each other. A branch channel can comprise linear areas, curved areas, or a combination thereof. The fluid processing device as provided that can comprise, for example, from one to twenty sets of aligned outlet chambers, from two to eighteen sets, from two to sixteen sets, from two to fourteen sets, from two to twelve sets, from two to ten sets, from two to eight sets, from four to six sets, two sets, four sets, or six sets, of aligned outlet chambers.

According to various embodiments, a fluid processing device is provided that can comprise two or more sets of fluid processing pathways. A set of fluid processing pathways can comprise, for example, two or more fluid processing pathways provided substantially parallel to and vertically offset from, a first set of fluid processing pathways that can comprise one or more, for example, two or more substantially parallel fluid processing pathways. For example, a fluid processing pathway of a first set can be vertically offset from a fluid processing pathway of a second set, such that the outlet chambers of the first set are vertically offset from the outlet chambers of the second set, and four sets of the outlet chambers can be formed. If the fluid processing device comprises a third set of offset fluid processing pathways, six sets of aligned outlet chambers can be formed. The distance between a first outlet chamber of a pathway of a member of a set of pathways and a second outlet chamber of that pathway, can be the same distance as the distance between the first outlet chamber of that pathway and the first outlet chamber of the next adjacent fluid processing pathway of that set, and can be the same as distance between a second outlet chamber of that pathway and the second outlet chamber of that next adjacent fluid processing pathway of that set. Aligned sets of outlet chambers can be configured, for example, spaced, to interface with a processing device comprising a plurality of ends arranged, for example, in an array. An aligned set of outlet chambers can comprise an array that can comprise aligned first outlet chambers and aligned second outlet chambers, that correspond to a set of fluid processing pathways. A plurality of arrays, each corresponding to a different set of fluid processing pathways, can be configured to interface with a processing device such that the ends of a processing device comprising a plurality of ends arranged in a first array, interface with a first array of outlet chambers. The ends of the processing device can then be removed from the outlet chambers of the first array, and moved into the outlet chambers of a second array of outlet chambers. Processing can continue in this manner for any number of outlet chamber arrays. The processing device can comprise, for example, a standard 1, 4, 16, 32, 48, or 96 capillary electrophoretic sequencer.

According to various embodiments, the processing device can comprise any piece of equipment for removing, storing, processing, and/or analyzing a fluid from a region, for example, a fluid contained in an outlet chamber. The outlet chambers can comprise, for example, an equidistant pitch, for example, a nine mm pitch. That is, the distance between each outlet chamber, for example outlet chambers A and B, in a specific fluid processing pathway and the distance between each of those outlet chambers A and B and each outlet chamber of the next adjacent pathway in that set of fluid processing pathways, for example, C and D, is such that the distance A-B, C-D, A-C, B-D, A-D, B-C, can be the same, for example, nine mm each. The distances between outlet chambers of a pathway and between pathways of a set, i.e., A-B and C-D, can be the same for each set of fluid processing pathways. Storage regions, if present, can also be configured as described, for example, two or more storage regions can be arranged on a nine mm pitch.

According to various embodiments, the distance between a fluid processing pathway of a first set and an immediately adjacent fluid processing pathway from a second set, can be less than the distance between a first and a second outlet chamber (A, B, respectively) in a fluid processing pathway and less than the distance between an outlet chamber, the first set and adjacent outlet chamber of an adjacent second set.

In some embodiments, each fluid processing pathway can comprise a linear series of multiple regions, that can optionally include one or more differently sized channels, for example, for connecting and/or blocking communication between adjacent regions. The regions, channels, or both, can each independently be empty, loaded with a reactant, agent, solution, or other material, or be provided with, for example, filtration media and/or frits. A fluid processing pathway can comprise an inlet chamber, and can comprise one or more reaction regions. Exemplary fluid processing devices can comprise a plurality of fluid processing pathways, for example, 48 , 64, 96, 128, 152, 192, or the like, fluid processing pathways, wherein each fluid processing pathway can comprise an independent inlet chamber, and one or more outlet chambers, for example, one or more dead-end outlet chambers. Each fluid processing pathway can comprise, for example, a split pathway, for example, that can branch into two or more branch channels. Two or more branch channels can be provided substantially parallel to each other. The two or more branch channels can be dead-end or open-ended.

In some embodiments, a fluid processing pathway can comprise one or more splitters to divide a sample through a series of regions wherein a portion of the sample continues along a first flow path or branch channel, and can involve, for example, a forward sequencing reaction, and the remainder of the sample can follow a second flow path or branch channel, and can involve a reverse sequencing reaction. In such splitting configurations, two respective outlet chambers, for example, dead-end outlet chambers, can be provided for analysis of forward-sequenced and reverse-sequenced products. The various regions can comprise different sizes and capacities. For example, a purification region can comprise a longer length and a larger capacity than a sequencing reaction region, and a polymerase chain reaction region can comprise double the capacity either of a forward-sequencing or a reverse-sequencing region. A PCR region can be provided in a fluid processing pathway, wherein the PCR region can be preloaded with PCR reactants sufficient to enable a desired amplification of a nucleic acid sequence.

A series of regions in a fluid processing pathway can comprise one or more purification regions, for example, a purification region provided downstream of a PCR region and provided upstream of one or more sequencing reaction regions. In some embodiments, a fluid processing device can comprise one or more purification regions that can be provided downstream of one or more respective sequencing reaction regions in a series of regions. If sequencing reaction regions are provided, they can be preloaded with sequencing reaction reactants that can enable a desired forward, reverse, or both forward and reverse sequencing reaction or group of reactions. Other pre-loaded components can comprise one or more of a buffer, a marker compound, a primer, and another component as would be recognized as suitable by those skilled in the art. The skilled artisan can readily select and employ suitable components for a desired reaction, without undue experimentation.

A fluid processing device can comprise different levels and layers of channels and regions. For example, a tiered, multi-channel device can comprise one or more fluid processing pathways that traverse different heights or levels in the substrate. In some embodiments a fluid processing device can comprise a tiered three-channel series. For example, a flow path can be manipulated from an inlet chamber to an outlet chamber, and can comprise a flow of fluid from the inlet chamber, through a lower channel, up a duct and through an upper channel, down a duct and through a second lower channel, and to one or more outlet chambers.

According to various embodiments, the fluid processing device can comprise a cover that can be provided on at least a portion of a top surface of a substrate. For example, the cover can be provided over the substrate such that one or more inlet chambers are left exposed. The cover can be provided over the substrate such that one or more outlet chambers are left covered and/or exposed. A cover layer can be provided that at least partially covers, one or more of a region, an inlet chamber, a channel, a duct, and the like.

A removable strip portion can then be provided over such exposed regions. The cover can comprise one or more cover portions, including, for example, a first cover portion covering a top surface of a proximal end of a substrate, wherein, for example, the cover ends at a center point of each aligned outlet chamber, and a second cover portion disposed over a top surface of a distal end of the substrate wherein the second portion ends at the center point of each aligned outlet chamber. The first and second cover portions can, for example, be provided such that they are in close proximity, contact each other, abut, or overlap. The cover can comprise additional portions corresponding to additional sets of, for example, aligned outlet chambers. If provided, the additional portion can correspond to a different set of aligned outlet chambers. A cover can comprise one or more of a permanently provided cover portion, a semi-permanently provided cover portion, a removably provided cover portion, and any combination thereof.

According to some embodiments, the cover can comprise a flexible material, a rigid material, an elastically deformable material, or a combination of two or more thereof. The cover can comprise a transparent, translucent or opaque material. The cover can comprise one or more of a color and a texture.

In some embodiments, the cover can comprise a flexible sheet. The cover can comprise an adhesive, flexible sheet. The cover can comprise a deformable, elastic cover. An elastically deformable cover layer can comprise PCR tape materials. Polyolefinic films, other polymeric films, copolymeric films, and combinations thereof can be used, for example, for an elastically deformable cover layer. The cover layer can comprise a thickness of, for example, from about 50 micrometers (μm) to about 100 μm.

If provided, the cover can comprise a gas permeable material. The cover can comprise one or more plastics. The cover can be permanently, semi-permanently, and/or removably provided on at least a portion of a top surface of a substrate, for example, by one or more of adhesive sealing, heat sealing, laminating, surface modification, ultrasonic sealing, chemical bonding, static forces, and the like. The cover can be provided on at least a portion of a top surface under conditions sufficient to form a fluid-tight seal.

According to some embodiments, a cover can be provided that comprises a non-porous, gas-permeable material cover and can comprise a cover layer that can be secured to the substrate by way of, for example, an adhesive, by heat bonding, by ultrasonic bonding, or by other application methods known to those of skill in the art. The cover layer can be hermetically sealed to an upper surface of the substrate. The cover layer can be in such intimate contact with the substrate that little, if any, leaking of an aqueous sample occurs between the cover layer and the substrate, for example, under a pressure of 50 psi water at 95° C. Gas contained in, or generated in, the reaction regions can be vented by molecular diffusion through the cover layer. The ready diffusion of gas through the cover layer can be provided by forming the cover layer of a polysiloxane material, for example, polydimethylsiloxane.

According to various embodiments, the cover layer can comprise a thickness of from about 0.001 inch to about 0.1 inch, for example, from about 0.003 inch to about 0.05 inch. Before, during, or after use, the fluid processing device can be further coated, sealed by, or covered by, or can be provided initially coated, sealed, or covered by, a gas-impermeable layer, for example, a non-porous aluminum film layer, a polyolefin film layer, or a polytetrafluoroethylene layer. The gas-impermeable layer can be capable of preventing evaporation, or other loss, or contamination, of a sample within the reaction region.

The non-porous, gas-permeable material of the cover can comprise, for example, a film, a sheet, or a strip, and can comprise at least one member selected from polysiloxane materials, polydimethylsiloxane materials, polydiethylsiloxane materials, polydipropylsiloxane materials, polydibutylsiloxane materials, polydiphenylsiloxane materials, and other polydialkylsiloxane or polyalkylphenylsiloxane materials. The polysiloxane can be the reaction product of an uncrosslinked reactive polysiloxane monomer and from about 0.01 weight percent to about 50 weight percent polysiloxane crosslinker, for example, from about 0.1 weight percent to about 25 weight percent or from about 0.5 weight percent to about 10 weight percent polysiloxane crosslinker.

The non-porous, gas-permeable material can comprise a polysiloxane material, a polyalkylsiloxane material, a polydialkylsiloxane material, a polyalkylalkylsiloxane material, a polyalklyarylsiloxane material, a polyarylsiloxane material, a polydiarylsiloxane material, a polyarylarylsiloxane material, a polycycloalkylsiloxane material, a polydicycloalkylsiloxane material, and combinations thereof. According to various embodiments, the polysiloxane material can include, for example, RTV 615, a polydimethylsiloxane material available from GE Silicones of Waterford, N.Y. The polysiloxane can be formed of a two-part silicone, for example RTV 615.

According to various embodiments, any suitable cover material can be utilized. Exemplary materials can comprise substantially chemically inert material, wherein the reagents can be pre-loaded into appropriate regions, for example, reaction regions of a fluid processing pathway. According to some embodiments, a cover can comprise a material that is capable of forming a substantially fluid-tight seal with the upper surface of the substrate, or appropriate regions thereof (e.g., an upstanding rim or lip about the opening of each reaction region). A seal can be affected, for example, using conventional adhesives and/or heat sealing techniques. Suitable heat-sealable materials can comprise, for example, polymeric films, such as polystyrene, polyester, polypropylene and/or polyethylene films. Such materials are available commercially, for example, from Polyfiltronics, Inc. (Rockland, Mass.) and Advanced Biotechnologies (Epsom, Surrey England UK).

According to various embodiments, a cover can comprise a substantially clear polymeric film, for example, being between about 0.05-0.50 millimeters thick, and that permits optical measurement of reactions taking place in the covered reaction regions. For example, real time fluorescence-based measurements of nucleic acid amplification products (such as PCR) can be obtained. In this technique, an excitation beam can be directed through the cover into each of a plurality of fluorescent mixtures separately contained in the reaction regions, wherein the beam has appropriate energy to excite fluorescent components in each mixture. Measurement of the fluorescence intensity indicates, in real time, the progress of each reaction. For purposes of permitting such real time monitoring, according to some embodiments, each cover can comprise a heat-sealable material that is transparent, or at least transparent at the excitation and measurement wavelength(s). A heat-sealable sheet can comprise a co-laminate of polypropylene and polyethylene. A heatable platen (not shown) can be used to engage the sheet, once cut and placed over an array of wells, and to apply heat so that the sheet bonds to the substrate.

Other exemplary cover layers that can be used include those described in U.S. patent application Ser. No. 10/762,786, filed Jan. 22, 2004, and in U.S. Patent Application Publication No. US 2003/0021734 A1, to VANN et al., filed Aug. 2, 2002, each of which is incorporated herein in its entirety by reference.

According to some embodiments, the fluid processing device can comprise an adhesive layer provided, for example, between the a top surface of a substrate and a lower surface of a cover, over a top surface of a substrate, over a lower surface of a cover, or any combination thereof. The adhesive can comprise an adhesive gasket layer provided between the substrate and the cover. The adhesive can comprise any suitable conventional adhesive. For example, an adhesive can comprise one or more of a permanent adhesive, a pressure-sensitive adhesive, a thermo-sensitive adhesive, and a non-permanent adhesive. Pressure sensitive adhesives can comprise one or more of silicone pressure sensitive adhesives, fluorosilicone pressure sensitive adhesives, and other polymeric pressure sensitive adhesives.

The adhesive can be provided over an entire surface or can be provided over at least a portion of a surface of, for example, a top surface of a substrate or a lower surface of a cover. For example, the adhesive can be provided over: all areas, including or corresponding to, recessed features; only on or corresponding to non-recessed features, wherein recessed features can comprise a region, a channel, a valve, and/or the like; or areas other than recessed and non-recessed areas comprising and surrounding a recessed feature, for example, an outlet chamber or regions. The adhesive can be provided over all areas with the exception of those areas that correspond to specific regions, areas, or channels, for example, outlet chambers. The adhesive layer can be provided over areas in the substrate corresponding to one or more outlet chambers, can be absent in areas corresponding to one or more outlet chambers, or can be provided over a portion of an area corresponding to one or more outlet chambers. The adhesive layer can comprise an adhesive gasket layer.

The adhesive can comprise any suitable thickness. The adhesive can be selected such that it does not deleteriously affect a sample, a desired reaction, or treatment of a sample processed through the fluid processing device. The adhesive layer can be more adherent to a cover, for example, an elastically deformable cover layer, than to the underlying substrate. An adhesive layer, if used, can be from about 50 μm to about 100 μm thick.

In some embodiments, an access area can comprise any area, configuration, or structure that allows access to a desired area, configuration, channel, duct, recess, region, structure, or the like. An access area can be provided to allow access to one or more regions, for example, one or more of an inlet chamber, a storage region, a reaction region, a purification region, a separation region, an incubation region, an outlet chamber, and the like. An access area can comprise an area that allows access to at least one or more outlet chambers.

In some embodiments, one or more access areas can be formed in a cover prior to providing the cover on a top surface of the substrate, or after the cover has been provided on the top surface of a substrate. In some embodiments, the access areas can be formed in a cover prior to providing the cover on the top surface of the substrate. Forming can comprise cutting slits.

In some embodiments, an access area can comprise one or more open areas not covered by the cover. Such open areas can be sealed with a removable strip. An access area can comprise, for example, a deformable opening that correspond to and allows access to, a region in a fluid processing pathway. An access area can comprise a flexible opening that deforms and allows entry of a structure, for example, one or more of ends of a processing device, for example, a capillary sequencing array, a pipette, a syringe, and the like. The deformable opening can comprise a slit. The slit can comprise a slit that completely or partially traverses the cover. The slit can be provided in the cover substantially perpendicular to the cover or at an angle relative to the cover. The slit can traverse only a partial thickness of the cover. The slit can comprise a continuous or discontinuous slit. A continuous slit can comprise a slit that does not comprise any uncut areas. A discontinuous slit can comprise uncut areas, for example, a linear or curved series of punctures, or a plurality of spaced apart slits radiating from an uncut center point.

In some embodiments, a slit can be made in the cover by any device capable of forming the slit including, for example, a cutting tool. The cutting tool can comprise a tool configured to provide a slit partially or completely traversing the cover, for example, a blade.

According to some embodiments, the cover can comprise a removable strip portion. The removable strip portion can comprise a flexible strip. The flexible strip can comprise plastic. The flexible strip can comprise a film or a non-permanent adhesive strip. The removable strip can be visually and/or tactically distinguishable from the fluid processing device and/or the cover. The removable strip can be optically clear, transparent, translucent, or opaque. The removable strip can comprise a color. The removable strip can comprise a texture. A cover can comprise one or more removable strips. A removable strip can be provided over the cover and can correspond to a set or sets of aligned regions, for example, aligned inlet chambers, or aligned outlet chambers.

In some embodiments, a system is provided that can comprise an apparatus, for example, a processing device, that can, for example, interface with two or more outlet chambers of a fluid processing device and can, for example, analyze, sequence, detect, or otherwise further treat, process, or manipulate a sample or product, for example, a reaction product, in a fluid processing device.

The analyzer can comprise a group of injectors, for example capillary injectors. The number of injectors in the group of injectors can be the same as, or less than, the number of outlet chambers in each group of outlet chambers. The injectors of the group of injectors can comprise respective distal tips arranged in a first array. The respective outlet chambers of each group of outlet chambers can be arranged in an array that matches the first array.

Various analyzers, detectors, and processors that can interface with two or more outlet chambers of a fluid processing device according to various embodiments, can comprise: separation devices, including electrophoretic, electroosmotic, or chromatographic devices; analyzing devices, including nuclear magnetic resonance (NMR) or mass spectroscopy devices; visualizing devices, including autoradiographic or fluorescent devices; recording or digitizing devices, such as a camera, a personal computer, a charged coupled device, or x-ray film; or any combination of the above apparati. An analyzing device can comprise, for example, a standard one, four, 16, 48, 96, or the like, capillary electrophoretic analyzers, for example, the Applied Biosystems 3730xL DNA analyzer, the Applied Biosystems 3730 DNA analyzer, the ABI PRISM 3100 genetic analyzer, the ABI PRISM 3100-Avant genetic analyzer, the Applied Biosystems 3130 genetic analyzer, the Applied Biosystems 3130xl genetic analyzer, the Applied Biosystems 310 genetic analyzer, and/or the ABI PRISM 7000 sequence detection system, all available from Applied Biosystems, Foster City, California (www.appliedbiosystems.com).

The analyzer can comprise a group of injectors, for example capillary injectors. The number of injectors in the group of injectors can be the same as, or less than, the number of outlet chambers in each group of outlet chambers. The injectors of the group of injectors can comprise respective distal tips arranged in a first array. The respective outlet chambers of each group of outlet chambers can be arranged in any array that matches the first array.

According to some embodiments, a system is provided that can comprise a support for supporting a fluid processing device, and a deformer that contacts the supported assembly and deforms at least one deformable valve (i.e., an intermediate wall), at least one deformable side wall, or any combination thereof, of the fluid processing device. According to some embodiments of the present teachings, the support, deformable valve, and deformer, can comprise those components described in co-pending United States Patent Application Publication No.: 2004/0131502 A1, to COX et al, filed Mar. 31, 2003, and in co-pending U.S. Patent Application Publication No.: 2004/0018116 A1, to DESMOND et al., filed Jan. 3, 2003, each of which is hereby incorporated by reference in its entirety, herein.

The inlet access area can be designed for loading a sample into a second region, for example, a reaction region, a purification region, a processing region, a separation region, an incubation region, an outlet chamber, or a storage region, by, for example, capillary action, gravity, or by force such as elevated pressure, and the like. An access area can be designed to enable venting of gas from a second region, which is displaced by sample that enters that region. An outlet access area can be designed to enable extraction of a sample from an outlet chamber.

In some embodiments, a method is provided for processing a sample. First, a sample reagent, or wash solution, can be dispensed into an inlet chamber of a fluid processing device. Dispensing can comprise manual dispensing, for example, using a standard multi channel pipette, or robotic dispensing using a robot, at any suitable time during the process, for example, at the beginning of the process. An inlet access area can be provided through a cover provided over the inlet chamber, or the inlet chamber can be exposed. A fluid sample can be moved from one region to an adjacent region through, for example, a channel, using, for example, one or more of centripetal force, pneumatic force, hydraulic force, vacuum, gravitational force, pressure, or the like. Spinning can be used to force fluid through, for example, a purification medium. Fluid communications, for example, via channels, between various regions can be selectively established by opening and closing (actuating) a valve provided between adjacent regions along a fluid processing pathway. After a valve is opened, fluid can be moved as described herein. Fluid mixing can comprise mixing by, for example, an external ultrasonic actuator or by oscillating a stepper motor. Time and temperature controls can be provided so that the fluid processing device can be subjected to an incubation period. Heating elements and cooling elements can be provided as part of a temperature control unit.

According to some embodiments, a method can comprise detecting a product processed in a fluid processing device. Detection can comprise detecting using a system according to some embodiments, or by implementing any of various independent detection systems.

Processed fluids can be preserved in the device, for example, in a storage region, stored, or removed from the device, for example, by pipetting or washing-out. For example, processed fluids can be stored in a storage region, for example, provided upstream from and adjacent to, a dead-end outlet chamber. The outlet chamber and the storage region can be separated by a valve.

Herein, a group of outlet chambers can comprise a plurality of outlet chambers each equally spaced from at least two other members of the group. For example, the outlet chambers can form a square matrix or a diamond matrix. What is meant by the phrase “between two or more outlet chambers of a different group of fluid processing pathways,” is that at least one outlet chamber of a first group of fluid processing pathways falls within the area outlined by the outlets of a different group of fluid processing pathways. In this way, the outlet chambers of two groups can have respective footprints that overlap one another. For example, if a first group of eight fluid processing pathways includes 16 outlet chambers arranged in two rows of eight, a second group of fluid processing pathways would include at least one outlet chamber disposed within the periphery defined by the 16 outlet chambers of the first group of fluid processing pathways.

According to various embodiments a fluid processing system can comprise a fluid processing device and an analyzer. The fluid processing device can comprise a substrate comprising a top surface and two or more sets of substantially parallel fluid processing pathways. Each fluid processing pathway can comprise at least one respective outlet chamber in communication with at least a portion of the top surface. Each set can comprise two or more fluid processing pathways or branch channels. The outlet chambers can be disposed in two or more groups. At least one of the outlet chambers of one of the sets of fluid processing pathways can be disposed between two or more outlet chambers of a group of fluid processing pathways.

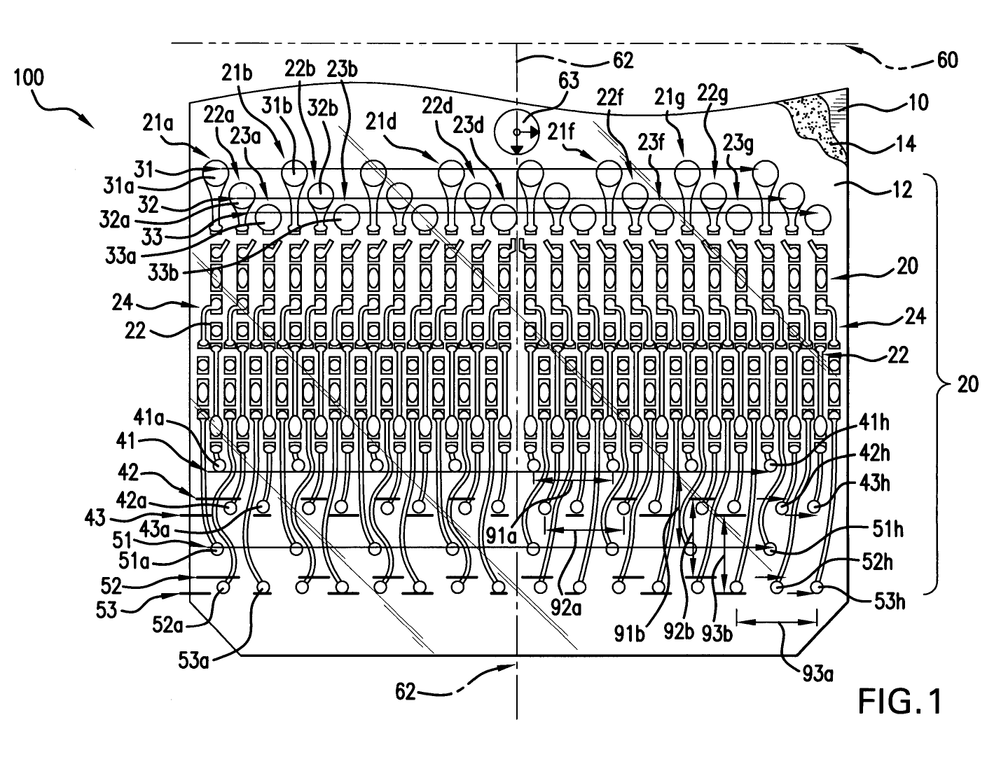

FIG. 1 illustrates a planar view of a fluid processing device 100 according to some embodiments. Fluid processing device 100 can comprise a substrate 10 that can comprise a plurality of substantially parallel fluid processing pathways, for example, split pathways 20, as shown. Split pathways 20 can be provided in communication with a top surface of the substrate 10. The device 100 can comprise a center opening 63 in the middle of which can be located a central axis of rotation. A cover 12 can be provided over at least a portion of the top surface of the substrate 10. An adhesive layer 14 can be provided between the top surface of the substrate 10 and a lower surface of the cover 12. Each split fluid processing pathway can comprise, for example, a teardrop-shaped inlet chamber 31a, a first branch channel 22, a first closed-end outlet chamber 41a, a second branch channel 24, and a second closed-end outlet chamber 51a. The plurality of split pathways can be provided substantially parallel to a vertical axis 62 of the device 100. In some embodiments, the cover 12 can be provided with a plurality of slits, holes, or other openings over or corresponding to inlet chambers, outlet chambers, or both. In some embodiments, the cover 12 is free of slits, holes, or other openings to the inlet chambers, the outlet chambers, or both.

The three sets of aligned outlet chambers 41, 42, and 43, the three sets of aligned outlet chambers 51, 52, and 53, and the three sets of aligned inlet chambers 31, 32, and 33, can be disposed in parallel to a horizontal axis 60, which is perpendicular to vertical axis 62.

FIG. 1 illustrates a plurality of fluid processing pathways 20 that can each comprise three groups of fluid processing pathways. For example, a first group 31 can comprise the set of split pathways 21a-21h, a second group 32 can comprise the set of split pathways 22a-22h, and a third group 33 can comprise the set of split pathways 23a-23h. Each group of split pathways can be vertically offset from the other groups of split pathways. This offset can result in a staggered pattern of input chambers such as that shown in input chambers 31a, 32a, and 33a. This staggered pattern can facilitate loading the split pathways.

The staggered pattern can configure the plurality of outlet chambers of each group to interface with an analyzer, for example, a 16-capillary, capillary electrophoretic analyzer. As illustrated, split pathway 21a can be provided at a first position and split pathway 22a can be provided adjacent to split pathway 21a but downwardly and vertically displaced from split pathway 21a. Likewise, the split pathway 23a can be provided adjacent to split pathway 22a but downwardly and vertically displaced from split pathway 22a. The second split pathway 21b of the first group 31 can be horizontally adjacent to split pathway 22a and can be at the same position relative to split pathway 21 a such that inlet chambers 31a and 31b are aligned, outlet chambers 41a and 41b are aligned, and outlet chambers 51a and 51b are aligned. Each aligned group of inlet chambers 31, 32, and 33, each aligned group of outlet chambers 41, 42, and 43, and each aligned group of outlet chambers 51, 52, and 53, is parallel or generally parallel to horizontal axis 60. Together, groups 41 and 51 define a first outlet chamber array, groups 42 and 52 define a second outlet chamber array. Together, groups 43 and 53 define a third outlet chamber array.

One outlet chamber of each group of fluid processing pathways can be spaced equidistant from an adjacent outlet chamber of an adjacent fluid processing pathway of that group.

In an exemplary embodiment, and as illustrated, the first outlet chamber 41a of split pathway 21a can be spaced apart from the second outlet chamber 51a of pathway 21a by a distance 91b. Likewise, outlet chamber 42a can be spaced apart from outlet chamber 52a by a distance 92b, and outlet chamber 43a can be spaced apart from outlet chamber 53a in pathway 23a at a distance 93b. The first outlet chamber 41a of split pathway 21a can be spaced apart from the first outlet chamber 41b (that is, the next adjacent outlet chamber of aligned outlet chambers 41) by a distance 91a. Likewise, outlet chambers 51a and 51b can be spaced apart by a distance 91a. The first outlet chamber 42a of pathway 22a can be spaced apart from the first outlet chamber 42b of pathway 22b by a distance 92a. The first outlet chamber 43a of pathway 23a can be spaced apart from the first outlet chamber 43b of pathway 23b by a distance 93a. Distances 91a and 91b can be equivalent, distances 92a and 92b can be equivalent, and distances 93a and 93b can be equivalent. Distances 91a, 92a, 91b, and 92b, can be equivalent. Distances 91a, 92a, 93a, 91b, 92b, and 93b, can be equivalent, for example, each distance can be 4.5, 5, 6, 7, 8, 9, 10, 11, 12, 13, 14, or 15 mm.

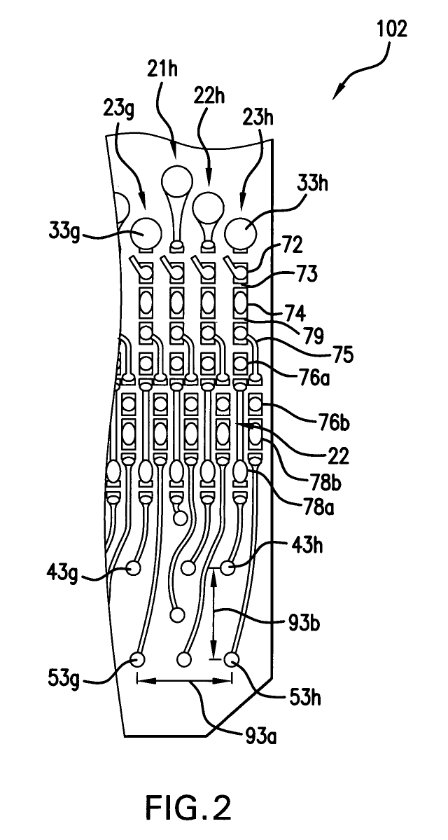

FIG. 2 is an enlarged, top plan view of a fluid processing device 102 similar to the fluid processing device illustrated in FIG. 1. A method for processing a liquid sample can comprise loading a liquid sample into aligned inlet chambers, for example, into the group of inlet chambers including inlet chambers 33g and 33h. Loading can be accomplished, for example, by using a standard multi-channel pipette wherein the inlet chambers can be configured to interface with the loading device. For example, a sample loaded into inlet chamber 33h can flow into, for example, a reaction region 72. Region 72 can be preloaded with PCR reagents, and PCR can be performed therein. A valve 73 can be actuated on the downstream side of region 72 and a PCR product can flow through the valve 73 from region 72 to a PCR purification region 74, for example, under the influence of centripetal force. Purification region 74 can contain purification media. After purification, a valve 79 can be actuated on the downstream side of purification region 74 and the purified PCR product can flow though a flow splitter 75. Flow splitter 75 can be configured such that a portion of the purified PCR product can flow to a preloaded forward sequencing reaction region 76a and another portion can flow to a preloaded reverse sequencing reaction region 76b.

Forward sequencing reaction region 76a and/or reverse sequencing reaction region 76b can be preloaded with respective forward and reverse sequencing reactants. Either or both of the forward and reverse sequencing reactions can be performed in either chamber or both chambers. After forward and reverse sequencing, sequencing reaction region valves can be opened on the downstream sides of the respective reaction regions, and the sequencing products can flow to forward sequencing purification region 78a and reverse sequencing purification region 78b, respectively. After purification, sequencing purification valves can be opened on the downstream sides of the respective purification regions. Thereafter, the purified forward sequencing reaction product can flow to first outlet chamber 43h and the purified reverse sequencing reaction product can flow to second outlet chamber 53h. An analyzer, for example, comprising a 16-capillary, capillary electrophoretic sequencing array, can then interface with aligned outlet chambers 43h and 53h, electrokinetically inject sequence reaction products contained in outlet chambers 43h and 53h, and analyze the products.

The liquid sample or a product of a reaction, can be caused to flow from one region, channel, or valve of the device, into an adjacent region, channel, or valve of the device, by, for example, centripetal force, capillary action, gravitational force, pneumatic force, pressure, hydraulic force, a combination of any two or more thereof, or the like.

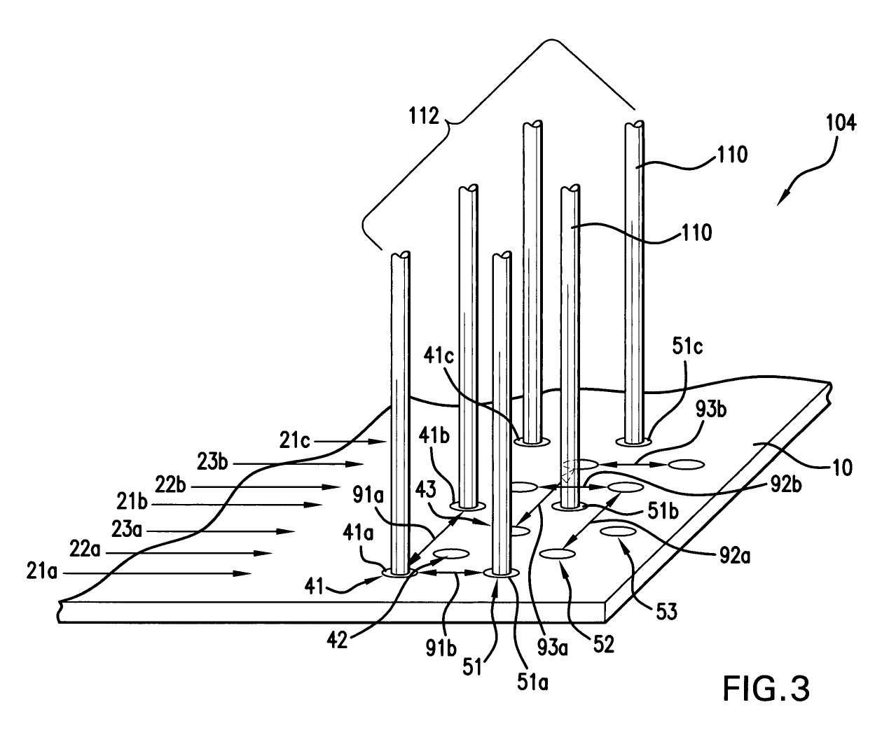

FIG. 3 illustrates a perspective view of a fluid processing device 104 and an analyzer 112 comprising a capillary injector array. The fluid processing device 104 can comprise a fluid processing device similar to either of those illustrated in FIGS. 1 and 2. Fluid processing device 104 can comprise a substrate 10 that can comprise three sets of substantially parallel fluid processing pathways 21, 22, and 23 (not shown), provided in communication with a top surface of the substrate 10. In FIG. 3, only the output regions of fluid processing pathways 21a, 21b, 21c, 22a, 22b, 23a, and 23b, are shown. Analyzer 112 can comprise any number of capillaries, for example, 4, 6, 8, 16, 48, 96, or the like, capillaries. Analyzer 112 can comprise at least six capillaries 110, as shown which interface respectively with output regions 41a, 41b, and 41c of aligned output regions 41, and with output regions 51a, 51b, and 51c of aligned output regions 51. The fluid processing pathways can be vertically offset as illustrated in FIGS. 1 and 2, such that the first outlet chambers 41, 42, and 43, and the second outlet chambers 51, 52, and 53, can be configured as shown. As shown in FIG. 3, distance 91a is the distance between the first outlet chambers 41a and 41b, and the distance between 41b and 41c, of the first three fluid processing pathways 21a, 21b, and 21c, of the first group of fluid processing pathways 21. Likewise, distance 91a is the distance between second outlet chambers 51a and 51b, and between 51b and 51c. Distance 91a can be equal to distances 92a and 93a, for example, to interface with an analyzer. An exemplary analyzer can be a multi-capillary, capillary electrophoretic sequencer. In the exemplary embodiment shown, distance 91b is the distance between the first outlet chamber 41a and the second outlet chamber 51a, and is also the distance between outlet chambers 41b and 51b, and is the distance between outlet chambers 41c and 51c. In some embodiments, distance 91b can be the same as distance 91a. In some embodiments, distance 91b can be the same as either or both distances 92b and 93b. In some embodiments, distances 91a, 91b, 92a, 92b, 93a, and 93b, can all be equivalent, for example, nine mm.

According to various embodiments, the distance between a fluid processing pathway of one group of pathways, and an adjacent fluid processing pathway of a different group of fluid processing pathways, can be selected to configure the outlet chambers described herein to interface with an analyzer, for example, a 1-, 4-, or 16-, capillary electrophoretic analyzer. A plurality of equally spaced outlet chambers can be considered a group of outlet chambers. The distance between adjacent fluid processing pathways of different groups, can also be selected sufficient to space apart aligned inlet chambers of pathways of a group of pathways, to interface with, for example, a standard multi-channel pipettor. Configurations, motions, and arrays of loading devices and inlet chambers can be provided, that are similar to those provided for the outlet chambers and processing device injectors. Fluid products contained in groups of outlet chambers of a group of fluid processing pathways, can be, for example, sampled, read, analyzed, removed, or the like, in groups that are linked to corresponding input regions.

Further fluid processing devices, substrates, covers, fluid processing manufacturing methods, input ports, output chambers, pathways, valves, reagents, flow restrictors, valve actuators, cutting tools, and methods of use are described in: U.S. Patent Application Publication No.: 2004/0131502, to COX et al., filed Mar. 31, 2003; U.S. Patent Application Publication No. 2004/0018116 to DESMOND et al., filed Jan. 3, 2003; U.S. Patent Application Publication No.: 2004/0055956 A1 to HARROLD, Michael P., filed on Jul. 28, 2003; U.S. Patent Application Publication No.: 20030152994, to WOUDENBERG et al., filed on Feb. 24, 2003; U.S. patent application Ser. No.: 11/029,968, filed on Jan. 5, 2005; and U.S. Patent Application Publication No.: 2004/0018117, to DESMOND et al., filed Jan. 3, 2003; each of which is hereby incorporated herein in its entirety by reference.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the present teachings be limited to the specific values recited when defining a range.

Other embodiments of the present teachings will be apparent to those skilled in the art from consideration of the present specification and practice of the present teachings disclosed herein. It is intended that the present specification and examples be considered as exemplary only.

What is claimed is:

1. A fluid processing system comprising:
a fluid processing device comprising a substrate comprising a top surface and two or more sets of fluid processing pathways, each fluid processing pathway comprising at least one respective outlet chamber in communication with at least a portion of the top surface, each set comprising two or more fluid processing pathways, wherein the outlet chambers are arranged in two or more arrays; and
an analyzer disposed on or adjacent to the substrate, the analyzer comprising a plurality of injectors, wherein the injectors comprise distal tips arranged in an array, the number of distal tips is the same as or less than the number of outlet chambers in one of the two or more arrays, and the distal tips are arranged in an array;
wherein each pattern formed by each respective outlet chamber array matches a pattern formed by the array of distal tips.

2. The fluid processing system of claim 1, wherein the array of distal tips is disposed in the outlet chambers of at least one of the two or more arrays of outlet chambers.

3. The fluid processing system of claim 1, further comprising a cover provided over at least a portion of the top surface, comprising one or more access areas, wherein each access area corresponds to one of the outlet chambers.

4. The fluid processing system of claim 1, wherein at least one of the fluid processing pathways comprises a reaction region.

5. The fluid processing system of claim 1, wherein at least one of the fluid processing pathways comprises a purification region.

6. The fluid processing system of claim 1, wherein at least one of the two or more sets comprises at least one valve disposed in fluid communication with at least one of the fluid processing pathways.

7. The fluid processing system of claim 1, wherein at least one of the two or more sets comprises at least one flow splitter disposed in fluid communication with at least one of the fluid processing pathways.

8. The fluid processing system of claim 1, wherein the outlet chambers of each array of outlet chamber are spaced apart from adjacent outlet chambers of the array by a first distance, and at least one of the two or more arrays of outlet chambers is offset from at least one other of the two or more arrays by a second distance that is less than the first distance.

9. The fluid processing system of claim 1, wherein an area outlined by a first array of the two or more arrays of outlet chambers overlaps an area defined by a second array of the two or more arrays of outlet chambers.

10. The fluid processing system of claim 1, wherein at least one of the two or more arrays of outlet chambers comprises at least 16 outlet chambers.

11. The fluid processing system of claim 1, wherein the analyzer comprises a multi-capillary, capillary electrophoretic analyzer.

12. A method, comprising:
providing a substrate comprising a top surface and two or more sets of fluid processing pathways, each fluid processing pathway comprising at least one respective outlet chamber in communication with at least a portion of the top surface, each set comprising two or more fluid processing pathways, wherein the outlet chambers are arranged in two or more arrays;
positioning an array of fluid injectors of an analyzer in the outlet chambers of a first array of the two or more arrays;
injecting respective samples disposed in the first array of outlet chambers into the analyzer; and
positioning the array of fluid injectors in the outlet chambers of a second array of the two or more arrays.

13. The method of claim 12, further comprising injecting a sample disposed in the second array of outlet chambers into the analyzer.

14. The method of claim 12, further comprising washing the array of injectors prior to inserting the injectors in the second array of outlet chambers.

15. The method of claim 12, further comprising, performing a sequencing reaction on the sample prior to injecting the sample into the analyzer.

16. The method of claim 12, further comprising:
loading one or more precursors into the plurality of fluid processing pathways; and
performing sequencing reactions on the one or more precursors to form the respective samples prior to injecting the respective samples into the analyzer.

17. The method of claim 12, wherein the analyzer comprises a multi-capillary, capillary electrophoretic analyzer.

18. The method of claim 17, further comprising electrophoretically separating the respective samples.

19. The method of claim 12, wherein each array of the two or more arrays comprises a rectangular footprint.

20. The method of claim 12, wherein the injecting respective samples comprises electrokinetically injecting the respective samples into a multi-capillary, capillary electrophoretic analyzer.

21. A fluid processing system comprising:
a fluid processing device comprising a substrate comprising a top surface and two or more sets of fluid processing pathways, each fluid processing pathway comprising at least one respective inlet chamber in communication with at least a portion of the top surface, each set comprising two or more fluid processing pathways, wherein the inlet chambers are arranged in two or more arrays; and
a loading device disposed on or adjacent to the substrate, the loading device comprising a plurality of injectors, wherein the injectors comprise distal tips arranged in an array, the number of distal tips is the same as or less than the number of inlet chambers in one of the two or more arrays, and the distal tips are arranged in an array;
wherein each pattern formed by each respective inlet chamber array matches a pattern formed by the array of distal tips.

22. A method, comprising:
providing a substrate comprising a top surface and two or more sets of fluid processing pathways, each fluid processing pathway comprising at least one respective inlet chamber in communication with at least a portion of the top surface, each set comprising two or more fluid processing pathways, wherein the inlet chambers are arranged in two or more arrays;
positioning an array of fluid injectors of a loading device in the inlet chambers of a first array of the two or more arrays;
injecting respective samples disposed in the first array of fluid injectors of the loading device into a first array of the two or more arrays; and
positioning the array of fluid injectors in the inlet chambers of a second array of the two or more arrays.
